(19) United States
(12) Patent Application Publication
(10) Pub. No.: US 2024/0130668 A1
(43) Pub. Date: Apr. 25, 2024

(54) DEVICE AND METHOD FOR MEASURING SKIN CHANGES CAUSED BY BLUE LIGHT, AND BLUE LIGHT IRRADIATION DEVICE

(71) Applicant: AMOREPACIFIC CORPORATION, Seoul (KR)

(72) Inventors: Hongli JO, Yongin-si (KR); Yuchul JUNG, Yongin-si (KR); Eun Joo KIM, Yongin-si (KR); Kyung Tae KIM, Seoul (KR); Yong Jin LEE, Namyangju-si (KR)

(73) Assignee: AMOREPACIFIC CORPORATION, Seoul (KR)

(21) Appl. No.: 18/391,138

(22) Filed: Dec. 20, 2023

Related U.S. Application Data
(62) Division of application No. 16/975,795, filed on Aug. 26, 2020.

(30) Foreign Application Priority Data
Feb. 26, 2018 (KR) 10-2018-0023179
Sep. 14, 2018 (KR) 10-2018-0110266

(51) Int. Cl. A61B 5/00; G16H 10/40; G16H 50/30

(52) U.S. Cl. CPC A61B 5/443; A61B 5/0082; A61B 5/4848; G16H 10/40; G16H 50/30; A61B 2562/0233

(57) ABSTRACT

The present invention relates to a device and a method for measuring skin changes caused by blue light. A device for measuring skin changes caused by blue light according to one embodiment of the present invention comprises: at least one light source for irradiating light of a blue light band at different light amounts to a plurality of test areas set on a skin area to be tested; and a control unit for detecting a minimum pigmentation point among the test areas and calculating a light amount of the corresponding test area as a minimum pigmentation dose (MPD) for blue light. In addition, the present invention relates to a blue light irradiation device for irradiating a plurality of blue light beams in order to measure skin changes caused by blue light.

### CROSS-REFERENCE TO RELATED APPLICATION

This application is a Divisional Application of U.S. patent application Ser. No. 16/975,795, filed Aug. 26, 2020, which is a National Stage Application of International Application No. PCT/KR2019/002293, filed Feb. 25, 2019, and claims priority to Korean Patent Application No. 10-2018-0023179, filed on Feb. 26, 2018 and Korean Patent Application No. 10-2018-0110266, filed on Sep. 14, 2018, and all the benefits accruing therefrom under 35 U.S.C. § 119, the contents of which are in their entirety are herein incorporated by reference.

### TECHNICAL FIELD

The present disclosure relates to a device and method for measuring skin changes caused by blue light and a blue light irradiation device, and more particularly, to a device and method for measuring skin changes caused by blue light, in which skin changes caused by blue light are measured, and further, the blue light blocking performance of blue light blocking products is measured, and a blue light irradiation device therefor.

### BACKGROUND ART

Exposure to light, such as exposure to sunlight, is a very important factor in terms of skin care and health. Excessive exposure to light may cause cell mutations or skin cancers due to ultraviolet (UV) radiation. In addition, excessive exposure to light causes skin loosening and skin spots, and thus has an important influence in terms of skin care.

Among various types of light, blue light is a short wavelength high energy visible light, it is emitted from sunlight, light emitting diode (LED) lights, TV and computer monitors and displays of smart devices, and it is known that blue light is harmful to human body.

Blue light has an adverse influence on human body, for example, dry eyes, reduced eyesight, reduced retinal function, and disturbs human biorhythm, causing disturbed sleep or reduced antioxidant capacity. Sunlight is only emitted during daytime, but people are exposed to blue light all day long due to their modern lifestyle, and exposure to blue light significantly affects health and skin care.

To measure the influence of blue light, in vitro tests such as cell tests and spectrum measurement are known. Patent Literature 1 discloses measuring the green response of skin to blue light and measuring an amount of elastotic materials, i.e., photodamage, based on the green response.

Patent Literature 1 only measures whether a responsive material is generated in response to blue light, and cannot measure skin changes occurred by blue light. That is, it is impossible to measure skin changes caused by blue light, such as pigmentation.

Additionally, blue light blocking products such as blue light shield products are available on the market, but there is no method for measuring or assessing the blue light blocking performance of the products. Accordingly, there has been no method for identifying or verifying the substantial effects when such products are applied to skin.

### RELATED LITERATURES

### Patent Literatures

(Patent Literature 1) U.S. Pat. No. 7,558,416 B2

### DISCLOSURE

### Technical Problem

The present disclosure is designed to solve the above-described problems, and therefore the present disclosure is directed to providing a device and method for measuring skin changes caused by blue light and a blue light irradiation device therefor.

The present disclosure is further directed to providing a device and method for measuring skin changes caused by blue light, in which the blue light blocking performance of blue light blocking products such as blue light shield products is measured and assessed, and a blue light irradiation device therefor.

The present disclosure is further directed to providing a device and method for measuring skin changes caused by blue light, in which skin changes are measured with changes of various factors such as the light intensity, the light amount and the irradiation time of blue light, and a blue light irradiation device therefor.

### Technical Solution

To achieve the above-described object of the present disclosure, a device for measuring skin changes caused by blue light according to an embodiment of the present disclosure includes at least one light source for irradiating light of a blue light band at different light amounts to a plurality of test areas set on a skin area to be tested, and a control unit for detecting a minimum pigmentation point among the test areas and calculating a light amount of the corresponding test area as a minimum pigmentation dose (MPD) for blue light.

The control unit may adjust either light intensity or irradiation time or both such that the light amount of the light source is larger at least twice than an average ultraviolet light amount.

The light source may be configured to irradiate light in a range between 450 and 470 nm.

The control unit may calculate and compare a first MPD and a second MPD of test areas to which a blue light blocking product to test is not applied and test areas to which the blue light blocking product to test is applied, respectively.

The blue light blocking product may be a blue light shield product, and the Protection grade of Blue-light (PB) of the blue light shield product may be calculated by [Equation 1]:

$$Protection grade of Blue - light ( PB ) = \frac{\begin{matrix}MPD of \\ skin with \\ blue light \\ blocking \\ product\end{matrix}}{\begin{matrix}MPD of \\ skin without \\ blue light \\ blocking \\ product\end{matrix}} \qquad [ Equation 1 ]$$

A method for measuring skin changes caused by blue light according to an embodiment of the present disclosure includes setting a plurality of test areas on a skin area to be tested, irradiating light of a blue light band at different light amounts to the test areas, detecting a minimum pigmentation point among the test areas and calculating a light amount of the corresponding test area as MPD for blue light.

Either light intensity or irradiation time or both may be adjusted such that the light amount of the blue light is larger at least twice than an average ultraviolet light amount.

The blue light band may range from 450 to 470 nm.

The minimum pigmentation point may be determined based on whether a contour of the test area is formed, whether pigmentation occurred at a preset area or more of the test area, or whether a change in melanin index before and after blue light irradiation is equal to or larger than a preset value or a preset percentage.

The method for measuring skin changes caused by blue light may include calculating a first MPD of test areas to which a blue light blocking product to test is not applied, and calculating a second MPD of test areas to which the blue light blocking product is applied.

The skin area may be a skin area of Fitzpatrick skin type III or above.

The blue light blocking product may be a blue light shield product, and the PB of the blue light shield product may be calculated by [Equation 1]:

$$Protection grade of Blue - light ( PB ) = \frac{\begin{matrix}MPD of \\ skin with \\ blue light \\ blocking \\ product\end{matrix}}{\begin{matrix}MPD of \\ skin without \\ blue light \\ blocking \\ product\end{matrix}} \qquad [ Equation 1 ]$$

A blue light irradiation device for irradiating a plurality of blue light beams according to an embodiment of the present disclosure includes a plurality of light sources to emit blue light and a plurality of glass fibers each connected to each light source, a light irradiation unit connected to each of the plurality of glass fibers and including a plurality of openings each for irradiating a concentric blue light beam, and a control unit to control power supplied to the plurality of light sources, wherein each of the plurality of light sources include a plurality of light emitting diodes (LEDs).

The control unit may control a light amount of each of the plurality of blue light beams by adjusting either power supplied to each of the plurality of light sources or irradiation time, or both.

Each of the plurality of light sources may be configured to irradiate light with peak wavelength of 456 nm and full width at half maximum (FWHM) of 21 nm.

The plurality of blue light beams irradiated through each of the plurality of openings may be simultaneously irradiated with each separate light intensity, irradiation time and irradiation type.

The irradiation type may be a pulse beam or continuous beam type.

The light irradiation unit may include six openings each for irradiating the blue light beam, and each of the plurality of light sources may include four mono-wavelength LEDs.

The blue light irradiation device may further include a heat sink and a cooling fan disposed around the plurality of light sources.

Each of the plurality of blue light beams irradiated through each of the plurality of openings may be such that blue light emitted from the plurality of LEDs is irradiated in a form of a concentric beam having a luminance difference of a reference value or less through each of the plurality of glass fibers.

### Advantageous Effects

According to an embodiment of the present disclosure, it is possible to provide a device and method for measuring skin changes caused by blue light that measures and analyzes the direct influence of blue light on skin, and a blue light irradiation device for irradiating a plurality of uniform blue light beams.

In addition, according to an embodiment of the present disclosure, it is possible to provide a device and method for measuring skin changes caused by blue light that measures and analyzes the blue light blocking performance of blue light blocking products such as blue light shield products by measuring skin changes caused by blue light, and a blue light irradiation device therefor.

Further, it is possible to provide a device and method for measuring skin changes caused by blue light that comprehensively analyzes the influence of blue light by measuring skin changes with changes of various factors such as the light intensity, the light amount and the irradiation time of blue light, and a blue light irradiation device with varying settings of various factors such as the light intensity or the irradiation time of a plurality of blue light sources.

### DESCRIPTION OF DRAWINGS

FIG. 1 is a schematic diagram of a device for measuring skin changes caused by blue light according to an embodiment of the present disclosure.

FIG. 2 is a schematic diagram of a method for measuring skin changes caused by blue light according to an embodiment of the present disclosure.

FIGS. 3A and 3B are images of test areas measured before and after blue light irradiation according to an embodiment of the present disclosure.

FIG. 4 is a graph showing the melanin index of test areas before and after blue light irradiation of FIGS. 3A and 3B.

FIG. 5 is a schematic diagram of a blue light irradiation device according to an embodiment of the present disclosure.

FIG. 6 is an enlarged view of a blue light irradiation area of a blue light irradiation device according to an embodiment of the present disclosure.

FIG. 7 shows a light source unit of a blue light irradiation device according to an embodiment of the present disclosure.

FIG. 8 shows a light connection unit of a blue light irradiation device according to an embodiment of the present disclosure.

FIG. 9 shows an example of connection between a light irradiation unit and a light source unit of a blue light irradiation device according to an embodiment of the present disclosure.

FIG. 10 is a bottom view of a light irradiation unit connected to a light source unit of a blue light irradiation device according to an embodiment of the present disclosure.

FIG. 11 is a graph showing changes in light intensity with changes in power level inputted to a blue light irradiation device according to an embodiment of the present disclosure.

FIGS. 12A to 12D show the beam uniformity measurement results with changes in power level inputted to a blue light irradiation device according to an embodiment of the present disclosure.

### BEST MODE

The embodiments may be modified in various forms and may have many embodiments, and particular embodiments will be illustrated in the drawings and described in detail. However, it is not intended to limit the scope to the particular embodiments, and it should be understood that the scope encompasses all alterations, equivalents or substitutes included in the spirit and technical scope disclosed herein. In describing the embodiments, when it is determined that a certain detailed description of relevant known technology may render the subject matter ambiguous, the detailed description is omitted herein.

In the embodiments, the ‘module’ or ‘unit’ performs at least one function or operation, and may be implemented in hardware or software or a combination of hardware and software. Additionally, except a ‘module’ or ‘unit’ that needs to be implemented in a specific hardware, a plurality of ‘modules’ or a plurality of ‘units’ may be integrated into at least one module and may be implemented as at least one processor (not shown).

Hereinafter, the embodiments will be described in detail with reference to the accompanying drawings, and in the description with reference to the accompanying drawings, like reference signs indicate like or corresponding elements and redundant descriptions are omitted herein.

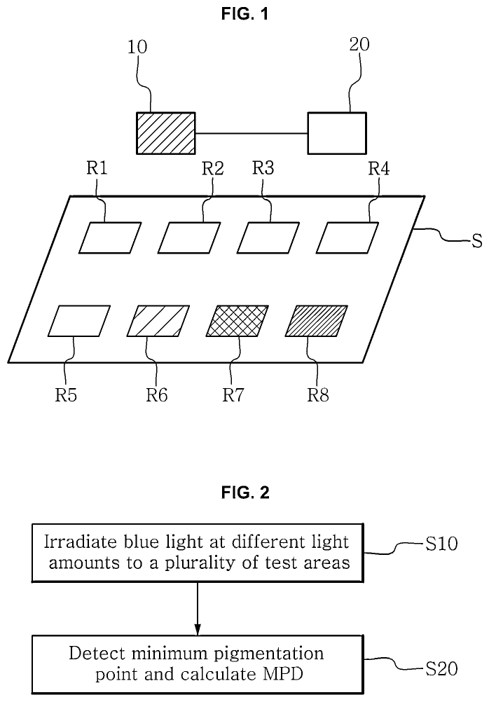

FIG. 1 is a schematic diagram of a device for measuring skin changes caused by blue light according to an embodiment of the present disclosure, and the device for measuring skin changes caused by blue light according to an embodiment includes at least one light source 10 to irradiate light of a blue light band at different light amounts onto a plurality of test areas, and a control unit 20 to detect a minimum pigmentation point among the test areas and calculate a light amount of the corresponding test area as a minimum pigmentation dose (MPD) for blue light.

The plurality of test areas R1, R2, R3, R4, R5, R6, R7, R8 is set on the skin area S at which skin pigmentation caused by blue light is to be measured. The skin area S may include a variety of skin areas such as arms, feet, face, back and neck. A mask (not shown) may be disposed at areas other than the plurality of test areas R1, R2, R3, R4, R5, R6, R7, R8 to irradiate blue light only within the test areas or to prevent the influence of the irradiated blue light on other test areas.

The light source 10 may be configured to irradiate light of a blue light band at different light amounts onto the plurality of test areas. The light amount may be expressed as the following [Equation 2].

Light amount (J/cm²)=Light intensity×Irradiation time [Equation 2]

To irradiate blue light at different light amounts onto the plurality of test areas R1, R2, R3, R4, R5, R6, R7, R8, the light intensity irradiated onto each test area R1, R2, R3, R4, R5, R6, R7, R8 may be adjusted, the irradiation time may be adjusted, and both the light intensity and the irradiation time may be adjusted.

The light source 10 corresponding to each test area R1, R2, R3, R4, R5, R6, R7, R8 may be disposed to control the light intensity irradiated onto each test area R1, R2, R3, R4, R5, R6, R7, R8.

That is, although one light source 10 is shown in the drawing, a plurality of light sources, for example, the corresponding number of light sources 10 to each test area R1, R2, R3, R4, R5, R6, R7, R8 may be included, but the present disclosure is not limited thereto.

Additionally, a mask may be further provided to shield each test area R1, R2, R3, R4, R5, R6, R7, R8 from blue light after a preset irradiation time, to control the irradiation time of each test area R1, R2, R3, R4, R5, R6, R7, R8.

Blue light refers to light having the wavelength in the range of about 400 to 495 nm. On the other hand, ultraviolet light has the range between about 10 and 400 nm. Since blue light has a longer wavelength than ultraviolet light, an amount of energy is smaller. Accordingly, it is difficult to measure the skin change characteristics by the same method.

Accordingly, to supply an amount of energy for inducing effective skin changes, according to an embodiment of the present disclosure, the control unit 20 may adjust either the light intensity or the irradiation time or both such that the light amount of the light source 10 is larger at least twice than the average ultraviolet light amount of about 27 J/cm². Specifically, the control unit 20 may adjust the light amount to about 55 J/cm² or more. When the light amount is less than twice, the amount of energy is small, so it is difficult to derive effective skin color changes, and errors caused by external impacts may increase.

Additionally, according to an embodiment of the present disclosure, the light source 10 may be configured to irradiate light of 450 to 470 nm. Light of less than 450 nm includes a violet range of wavelengths, and thus it is difficult to measure the influence of pure blue light, and light of more than 470 nm includes a green range of wavelengths, and thus, likewise, it is difficult to measure the influence of pure blue light, causing an error.

The light source 10 may include, but is not limited to, a blue light emitting diode (LED) source.

Additionally, according to an embodiment of the present disclosure, the control unit 20 detects a minimum pigmentation point among the test areas, and calculates the light amount of the corresponding test area as MPD for blue light.

Blue light has a longer wavelength than UVA, and thus may induce darkening to skin. The control unit 20 detects a minimum pigmentation point at which darkening is induced among the plurality of test areas R1, R2, R3, R4, R5, R6, R7, R8, and detects a minimum pigmentation point at which darkening occurred at a minimum light amount.

Additionally, to derive effective skin changes caused by blue light, subjects having Fitzpatrick skin type (see [Table 1] below) III or above, prone to pigmentation, may be selected. It will be helpful in obtaining accurate results by easily deriving effective skin changes when testing the blue light blocking performance of blue light blocking products.

TABLE 1

| Fitzpatrick | |
|---|---|
| skin type | Description |
| 1 | Burns easily, does not tan |
| 2 | Burns easily, tans minimally |
| 3 | Mild burn, tans gradually |
| 4 | Rarely burns, tans gradually |
| 5 | Rarely burns, tans pretty darkly |
| 6 | Never burns, tans very darkly |

FIG. 1 shows an embodiment in which the light amount increases in a sequential order of the plurality of test areas R1, R2, R3, R4, R5, R6, R7, R8, and in this case, darkening may be induced in three test areas R6, R7, R8 as in FIG. 1. Among the three test areas R6, R7, R8, the test area R6 at which darkening occurred at the minimum light amount is a minimum pigmentation point, and the light amount at the test area R6 is MPD.

According to an embodiment of the present disclosure, skin changes caused by blue light on the target skin may be measured by calculating the MPD value which is the light amount of the test area at which effective skin changes caused by blue light occurred. For example, as the skin or skin area has larger MPD, the skin or skin area may have lower sensitivity to blue light.

Meanwhile, according to an embodiment of the present disclosure, it is possible to measure the blue light blocking performance or effect of blue light blocking products. Since blue light is not only included in the visible range of sunlight, but also is emitted from LED lights, LED displays, smartphones, tablets and monitors, people are always exposed to blue light in daily life. Accordingly, information associated with the blue light blocking performance of blue light blocking products may be an important indicator to consumers to buy or use blue light blocking products.

According to an embodiment of the present disclosure, it is possible to conduct an intensive analysis on the blue light blocking effect corresponding to how much the influence on skin is substantially reduced by blue light blocking products.

To this end, the control unit 20 may compare and derive MPDs of test areas with and without a blue light blocking product to test.

To this end, the control unit 20 may calculate and compare a first MPD and a second MPD of test areas with and without a target blue light blocking product to test, respectively.

The blue light blocking product is a blue light blocking cosmetic product. The present disclosure may provide the blue light blocking performance of the blue light blocking product applied to skin quantitatively/numerically to inform or verify the blue light blocking performance of the product.

Specifically, the Protection grade of Blue-light (PB) of the blue light blocking product to test may be calculated by [Equation 1].

$$Protection grade of Blue - light ( PB ) = \frac{\begin{matrix}MPD of \\ skin with \\ blue light \\ blocking \\ product\end{matrix}}{\begin{matrix}MPD of \\ skin without \\ blue light \\ blocking \\ product\end{matrix}} \qquad [ Equation 1 ]$$

The Protection grade of Blue-light (PB) indicates the blue light blocking effect of the blue light blocking product, and as the grade is higher, the blue light blocking effect of the blue light blocking product is better.

That is, it is possible to inform how much the effect on skin pigmentation is blocked by measuring a ratio of the MPD of the skin with the blue light blocking product and the MPD of the skin without the blue light blocking product.

FIG. 2 is a schematic diagram of a method for measuring skin changes caused by blue light according to an embodiment of the present disclosure. The device for measuring skin changes may be applied to the method for measuring skin changes. Accordingly, the description of the device for measuring skin changes may be applied to the method for measuring skin changes or vice versa.

Referring to FIG. 2, the method for measuring skin changes caused by blue light includes a first step S10 of irradiating light of a blue light band at different light amounts onto a plurality of test areas set on a skin area, and a second step S20 of calculating a light amount of a minimum pigmentation point among the test areas as MPD for blue light.

The light amount of the blue light source may be set to irradiate different light amounts onto each test area. As the units of light amount, light intensity and irradiation time of the blue light source are not set or known, the unit of light intensity may be determined as the unit of a detector, W/cm², and then the units of light amount and irradiation time may be set.

As each light amount is determined by the light intensity and the irradiation time as described above in the device for measuring skin changes, two methods may be used; adjusting the irradiation time while irradiating blue light of the same light intensity onto the skin, or irradiating blue light of different light intensities for the same irradiation time. The light amount may be adjusted by simultaneously adjusting the light intensity and the irradiation time.

The upper and lower limits of the light amount of blue light may be differently set depending on subjects or skin areas. However, to derive effective skin changes, either the light intensity or the irradiation time or both may be adjusted such that the upper and lower limits are higher at least twice than the average ultraviolet light amount. Since energy of blue light is lower than that of ultraviolet light, the light amount required to cause skin pigmentation may be set larger.

According to an embodiment, the blue light band may be 450 to 470 nm. It is to prevent an error by preventing the influence of violet light or green light.

Additionally, since blue light has a longer wavelength than UVA, considering that darkening will be induced, subjects having Fitzpatrick skin type III or above, prone to pigmentation, may be selected. It is to smoothly induce pigmentation.

The minimum pigmentation point is a point having a minimum pigmentation dosage where darkening occurred, and the point at which darkening occurs for the first time may be determined based on whether the contour of the test area clearly appeared, whether pigmentation occurred at a preset area or more of the test area, or a difference in melanin index before and after blue light irradiation is equal to or larger than a preset value or a preset percentage.

More specifically, whether the contour of the pigmented and darkened area is clear may be detected, whether pigmentation occurred at an area of ⅔ or more of the test area may be detected, or the melanin index may be measured using an instrument and a test area having a difference equal to or larger than a preset value may be detected.

In the case of the melanin index, a test area at which pigmentation occurred for the first time may be detected by determining a difference in melanin index before and after blue light irradiation. Accordingly, a point at which a difference in melanin index occurred for the first time may be detected as the minimum pigmentation point, and MPD may be determined. However, the present disclosure is not limited thereto, and even when there is a difference in melanin index, there may be an area at which darkening is invisible, and thus a point at which a difference in melanin index is equal to or larger than a preset value or a preset percentage may be detected and set as the minimum pigmentation point, and MPD at the point may be determined.

The minimum pigmentation point may be detected by the above-described method, and skin changes caused by blue light and the blue light blocking effect of the blue light blocking product may be discerned by deriving the light amount at the minimum pigmentation point.

Additionally, according to an embodiment of the present disclosure, to measure and verify the blue light blocking effect of the blue light blocking product to test, a first MPD may be calculated by performing the first step S10 and the second step S20 on test areas to which the blue light blocking product to test is not applied, and a second MPD may be calculated by performing the first step S10 and the second step S20 on test areas to which the blue light blocking product is applied.

According to an embodiment, the second MPD may be calculated for the skin to which the blue light blocking product is applied at a light amount that is larger twice to four times than the first MPD.

According to the above-described method, it is possible to verify the blue light blocking performance or effect of the blue light blocking product by comparing the first and second MPDs before and after applying the blue light blocking product.

According to an embodiment, the blue light blocking effect of the blue light blocking product may be calculated as the Protection grade of Blue-light (PB), and may be calculated by [Equation 1] below.

$$Protection grade of Blue - light ( PB ) = \frac{\begin{matrix}MPD of \\ skin with \\ blue light \\ blocking \\ product\end{matrix}}{\begin{matrix}MPD of \\ skin without \\ blue light \\ blocking \\ product\end{matrix}} \qquad [ Equation 1 ]$$

Accordingly, it is possible to verify the blue light blocking effect of the blue light blocking product based on skin changes caused by blue light, and provide its difference as quantitative data.

Further, it is possible to directly assess and represent the effect of the blue light blocking product. Additionally, it can be used as an evidence for demonstrating the blue light blocking performance of the blue light blocking product.

According to various embodiments of the present disclosure, it is possible to observe skin changes caused by blue light by the application of different light amounts for each skin area. As the device and method for measuring skin changes according to the present disclosure is possible in vivo testing, it is possible to measure the direct influence of blue light on skin changes. That is, as the influence of blue light on human body is measured in terms of pigmentation, it is possible to verify, measure and compare the substantial influence of blue light on skin.

Additionally, it is possible to set and change many factors that affect blue light measurement, such as the light intensity, the light amount and the irradiation time of blue light and subject selection, thereby providing the device and method for measuring skin changes that can conduct analysis in various aspects.

The light amount setting method may include adjusting the irradiation time while irradiating the blue light source of the same light intensity onto the skin, or irradiating the blue light source of different light intensities for the same irradiation time. These methods may adjust the total light amount, and measure skin pigmentation caused by blue light differently formed from the adjusted light amount.

According to the conventional art, the basis for demonstrating that blue light blocking products actually block blue light is insufficient, and it is difficult to provide information about how the products block blue light and the extent to which the products block blue light. However, according to various embodiments of the present disclosure, it is possible to assess the influence of blue light on skin, and based on this, demonstrate the blue light blocking effect of blue light blocking products, in particular, blue light shield products, and provide quantitative information about the blue light blocking effect.

In addition, it can be used as data for demonstrating the efficacy of blue light blocking products, and it is possible to analyze various characteristics of blue light blocking products.

### Embodiment 1

For six test areas T1, T2, T3, T4, T5, T6, different light amounts are set and irradiated as shown in the following [Table 2].

TABLE 2

| Test area | Light amount |
|---|---|
| T1 | 5 J/cm² |
| T2 | 10 J/cm² |
| T3 | 30 J/cm² |
| T4 | 60 J/cm² |
| T5 | 80 J/cm² |
| T6 | 90 J/cm² |

Darkening is induced with different light amounts in each test area by setting the lowest light amount of the test area T1 and increasing the light amount toward the test area T6.

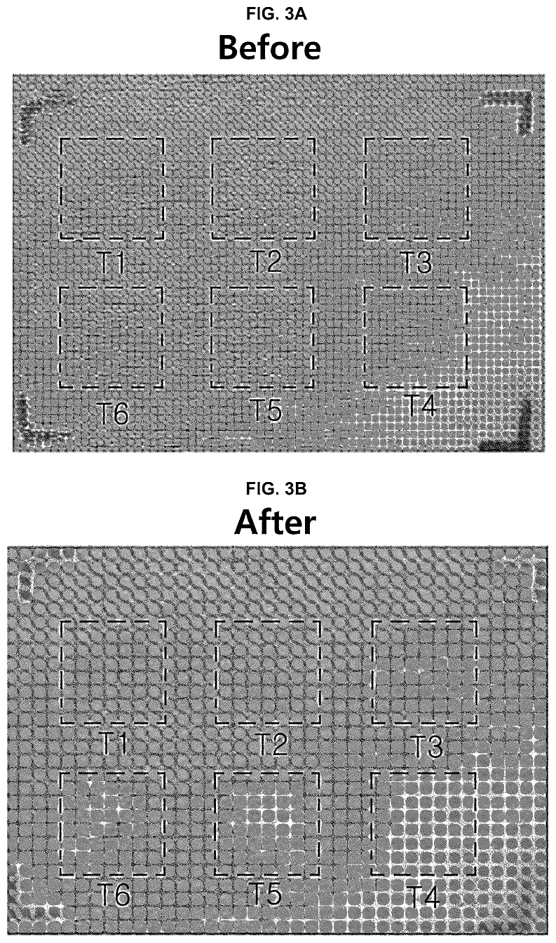

Images of the test areas before blue light irradiation (A) and immediately after blue light irradiation (B) are shown in FIGS. 3A and 3B. Referring to FIGS. 3A and 3B, the contour of each area is clearly seen and darkening is observed in the test areas T4, T5, T6. Among the test areas T4, T5, T6, the test area T4 has the minimum light amount, and thus it can be seen that MPD is 60 J/cm².

### Embodiment 2

For six test areas T1, T2, T3, T4, T5, T6 that are different from those of Embodiment 1, different light amounts are set and irradiated as shown in the above [Table 2].

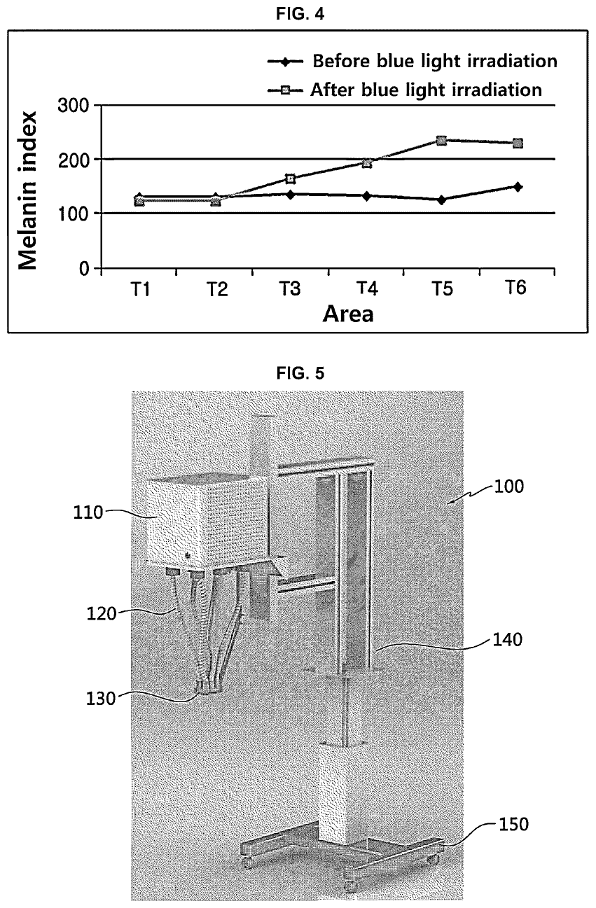

FIG. 4 is a graph showing the melanin index measured before and immediately after blue light irradiation on the six test areas T1, T2, T3, T4, T5, T6 of Embodiment 2.

Referring to FIG. 4, seeing a difference in melanin index before and after blue light irradiation for each test area, it can be quantitatively seen that the melanin index increases from the test area T3 and pigmentation occurred.

The test area T3 has an increase in melanin index of about 17% or more, and the test area T4 has an increase in melanin index of about 30% or more. A change in melanin index before and after blue light irradiation shows skin changes depending on the amount of blue light. Through the increase in melanin index, it is possible to provide quantitative data about how much pigmentation occurs according to the amount of blue light, and it can be used as data for determining MPD.

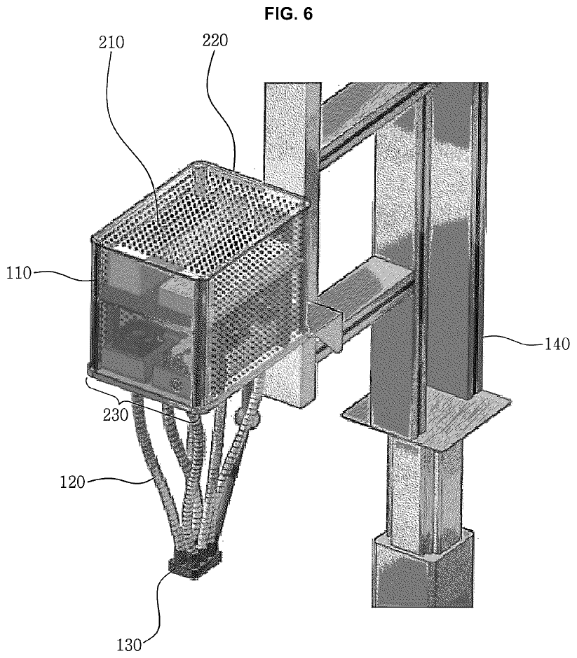

FIG. 5 is a schematic diagram of a blue light irradiation device 100 according to an embodiment of the present disclosure. FIGS. 6 to 10 are detailed diagrams of each element of the blue light irradiation device according to an embodiment of the present disclosure.

Referring to FIGS. 5 and 6, the blue light irradiation device 100 according to an embodiment of the present disclosure includes a light source unit 110, a cable 120, a light irradiation unit 130, a fixation unit 140 and a support unit 150.

As shown in FIG. 6, for example, the light source unit 110 of the blue light irradiation device 100 may include a control unit 210, a light source array 230 and a cover 220.

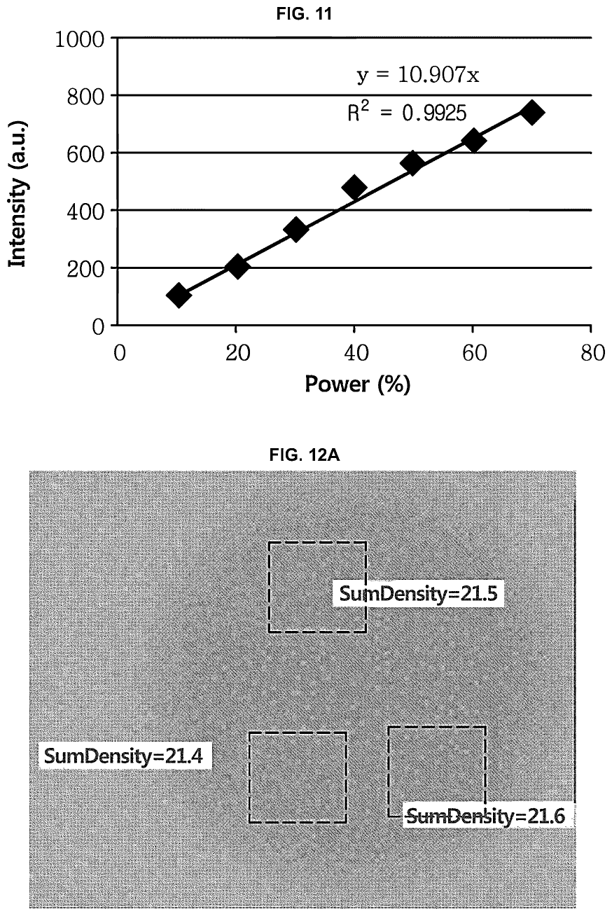

The control unit 210 may control the level and duration of power supplied to the light source array 230 that emits blue light. As shown in FIG. 11, since the light intensity of blue light is approximately proportional to power, when the level and duration of supplied power is controlled, the light intensity and light duration of blue light may be controlled, and through this, the light amount of blue light may be controlled. The light amount may be expressed as [Equation 2] below.

Light amount (J/cm²)=Light intensity×Irradiation time [Equation 2]

The control unit 210 may independently control the light intensity and the irradiation time of blue light beams irradiated by each of a plurality of openings 131 as described below. Through this, it is possible to simultaneously irradiate each blue light beam of different light intensities, thereby conveniently measuring the influence of blue light (for example, skin changes, skin pigmentation, etc.).

It is possible to independently set the light intensity and the irradiation time of each of the plurality of blue light beams by an interface (not shown) connected to the control unit 210. The interface may include at least one of a touch pad, an input button or a display.

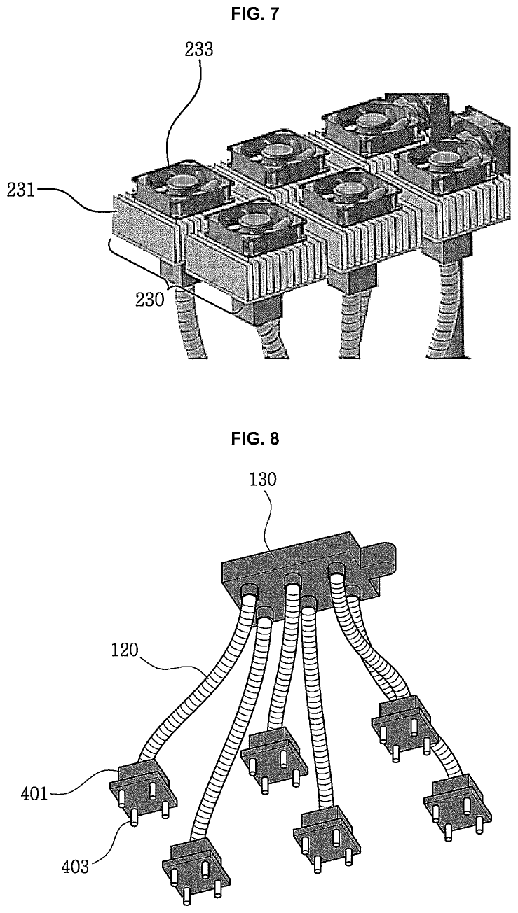

As shown in FIGS. 6 and 7, the light source array 230 includes a plurality of light sources 231. For example, the light source array 230 may include six light sources 231, and a heat sink and a fan type cooling system such as a cooling fan 233 to properly remove the unsafety of temperature changes near the plurality of light sources 231.

Each light source 231 may include at least one LED chip (not shown). According to an embodiment of the present disclosure, each light source 231 may include a plurality of LED chips to further increase the light intensity, thereby achieving high light intensity irradiation in a short time, resulting in reduced clinical test time required to measure changes caused by blue light irradiation. For example, each light source 231 may include four LED chips, and it is possible to irradiate blue light of the light intensity that is higher about 4 times than the light intensity when one LED chip is used.

The control unit 210 and the light source array 230 may be wrapped around the cover 220, and the cover 220 may include a plurality of openings to make it easy to release heat as shown in FIGS. 5 and 6.

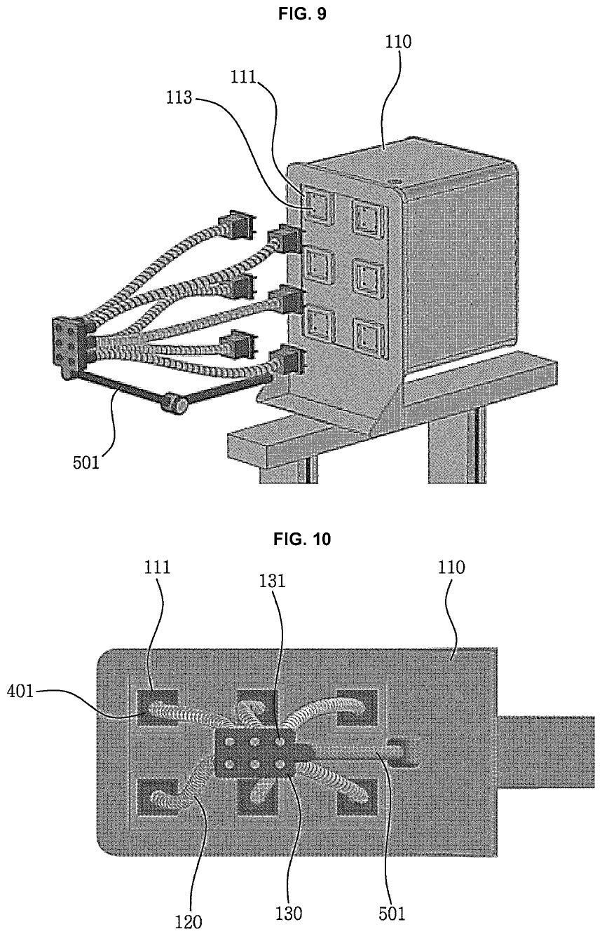

As shown in FIG. 9, the light source unit 110 may include, on the lower surface, a coupler 111 to connect the light source unit 110 to the cable 120 made of glass fiber to emit blue light emitted from the plurality of LED chips as a concentric beam. According to an embodiment of the present disclosure, a plurality of glass fiber cable 120 may be each connected to the plurality of light sources 231 included in the light source array 230 to simultaneously irradiate the plurality of blue light beams.

The plurality of glass fiber cables 120 may be each connected to the plurality of corresponding couplers 111 to correct blue light emitted from the plurality of LED chips to a concentric beam. As shown in FIG. 8, a coupler 401 for connection to the light source unit 110 is included at one end of each of the plurality of cables 120, and the coupler 401 includes the corresponding number of male connectors 403 to the number of channels. For example, as shown in FIG. 8, when each light source 231 includes four LED chips, the 4-channel cable 120 may include four male connectors 403 at the coupler 401. The four male connectors 403 disposed at the coupler 401 of each cable 120 may receive blue light emitted from the four LED chips when connected to four female connectors 113 disposed at the coupler 111 of the light source unit 110 in the same way.

The other end of the plurality of glass fiber cables 120 is connected to the light irradiation unit 130. As shown in FIG. 10, the light irradiation unit 130 may include a plurality of openings 131 to irradiate each of the plurality of blue light beams emitted from the plurality of corresponding cables 120. For example, six openings 131 each connected to six cables 120 may be configured to simultaneously irradiate six blue light beams.

As shown in FIGS. 9 and 10, the light irradiation unit 130 may be fixedly connected to the light source unit 110 through a holder 501. The holder 501 may have an adjustable length to adjust the distance between the light irradiation unit 130 and the light source unit 110 and maintain the adjusted distance.

The light source unit 110 may be connected to the fixation unit 140 that forms the body of the blue light irradiation device 100, and as shown in FIG. 5, may be fixed at a predetermined height from the ground.

The fixation unit 140 may extend to the ground and be connected to the support unit 150. The support unit 150 is connected to the fixation unit 140 to form the body of the blue light irradiation device 100 as a whole, and serves as a support to stably maintain the blue light irradiation device 100. According to an embodiment of the present disclosure, the support unit 150 may include a moving means (for example, wheels) to facilitate the movement of the blue light irradiation device 100.

Using the blue light irradiation device 100, it is possible to simultaneously irradiate blue light beams of different light amounts onto a plurality of areas and easily observe the resulting changes. Particularly, it is possible to test in various conditions by independently controlling the light intensity or the irradiation time while simultaneously irradiating the plurality of blue light beams.

According to an embodiment of the present disclosure, using the blue light irradiation device 100, it is possible to irradiate blue light onto human body, in particular, skin, and measure skin changes and assess the influence on skin.

For example, to measure skin pigmentation cause by blue light, the light irradiation unit 130 of the blue light irradiation device 100 may be disposed in contact with or near the plurality of test areas to irradiate the blue light beams. The skin area to be measured may include a variety of skin areas such as arms, feet, face, back and neck. Particularly, as the cable 120 of the blue light irradiation device 100 can flexibly deform, it is possible to irradiate a desired light amount of blue light onto a desired area of the curved human body such as back and waist by appropriately adjusting the position of the light irradiation unit 130. According to an embodiment of the present disclosure, the angle (90° or higher) and position of the light irradiation unit 130 of the blue light irradiation device 100 can be adjusted to suit for human tests.

According to an embodiment of the present disclosure, when a blue light irradiation test is conducted with the light irradiation unit 130 in contact with the skin, it is possible to prevent the irradiation of blue light to areas other than the areas corresponding to the openings 131 of the light irradiation unit 130.

To supply an amount of energy for inducing effective skin changes, according to an embodiment of the present disclosure, the blue light irradiation device 100 may adjust either the light intensity or the irradiation time or both such that the light amount of the light source 231 is larger at least twice than the average ultraviolet light amount of about 27 J/cm². Specifically, the light amount may be adjusted to about 55 J/cm² or more. When the light amount is less than twice, the amount of energy is small, so it is difficult to derive effective skin color changes, and errors caused by external impacts may increase.

Additionally, according to an embodiment of the present disclosure, the light source 231 of the blue light irradiation device 100 may be configured to irradiate light of peak wavelength of 456 nm and full width at half maximum (FWHM) of 21 nm. Light or less than 450 nm includes a violet range of wavelengths, and thus it is difficult to measure the influence of pure blue light, and light of more than 470 nm includes a green range of wavelengths, and thus, likewise, it is difficult to measure the influence of pure blue light, causing an error.

The light source 231 of the blue light irradiation device 100 may include, but is not limited to, a blue LED source.

According to an embodiment of the present disclosure, to measure skin pigmentation using the blue light irradiation device 100, a minimum pigmentation point may be detected and a light amount of the corresponding test area may be calculated as MPD for blue light. Specifically, since blue light has a longer wavelength than UVA, blue light may induce darkening to the skin, detect a minimum pigmentation point among the plurality of test areas (for example, six test areas) and calculate the light amount of the corresponding test area as MPD for blue light.

According to an embodiment of the present disclosure, an increase in melanin index may be measured in the plurality of areas irradiated with blue light by the blue light irradiation device 100, and quantitative data about to which extent pigmentation occurred may be calculated according to an amount of blue light. This may be used as data for determining MPD.

Additionally, to derive effective skin changes for blue light, subjects having skin type (for example, Fitzpatrick skin type III or above) prone to pigmentation may be selected.

According to an embodiment of the present disclosure, it is possible to measure the blue light blocking performance or effect of blue light blocking products by easily deriving effective skin changes caused by blue light using the blue light irradiation device 100 as described above. Since blue light is not only included in the visible range of sunlight, but also is emitted from LED lights, LED displays, smartphones, tablets and monitors, people are always exposed to blue light in daily life. Accordingly, information associated with the blue light blocking performance of blue light blocking products may be an important indicator to consumers to buy or use blue light blocking products.

According to an embodiment of the present disclosure, it is possible to conduct an intensive analysis on the blue light blocking effect corresponding to how much the influence on skin is reduced by blue light blocking products.

To this end, using the blue light irradiation device 100, it is possible to compare and derive the MPDs of test areas with and without a blue light blocking product to test.

The blue light blocking product may be, for example, a blue light blocking cosmetic product. The present disclosure may provide the blue light blocking performance of the blue light blocking product applied to skin quantitatively/numerically to inform or verify the blue light blocking performance of the product.

Specifically, the Protection grade of Blue-light (PB) of the blue light blocking product to test may be calculated by [Equation 1] as described above. In this way, it is possible to quantitatively assess the blue light blocking performance of the blue light blocking product using the blue light irradiation device 100.

$$Protection grade of Blue - light ( PB ) = \frac{\begin{matrix}MPD of skin \\ with blue \\ light blocking \\ product\end{matrix}}{\begin{matrix}MPD of skin \\ without blue \\ light blocking \\ product\end{matrix}} \qquad [ Equation 1 ]$$

According to the conventional art, the basis for demonstrating that blue light blocking products actually block blue light is insufficient, and it is difficult to provide information about how the products block blue light and the extent to which the products block blue light. However, using the blue light irradiation device 100 that simultaneously irradiates blue light onto a plurality of areas according to various embodiments of the present disclosure, it is possible to assess the influence of blue light on skin, and based on this, demonstrate the blue light blocking effect of blue light blocking products, in particular, blue light shield products, and provide quantitative information about the blue light blocking effect.

The blue light irradiation device 100 may be implemented to simultaneously irradiate the blue light beams of different light amounts on the plurality of areas to measure the influence of blue light, in particular, skin changes, using the blue light irradiation device 100.

The light amount of the blue light beam of the blue light irradiation device 100 may be controlled by adjusting the light intensity or the irradiation time, or changing the settings of the light intensity and the irradiation time.

FIG. 11 shows a graph showing changes in light intensity with the changes in power level inputted to the blue light irradiation device 100 according to an embodiment of the present disclosure. As shown in FIG. 11, it can be seen that when the power level changes, the light intensity of blue light changes approximately in proportion thereto.

According to an embodiment of the present disclosure, the blue light irradiation device 100 may be adjusted to the maximum of 8 W/cm².

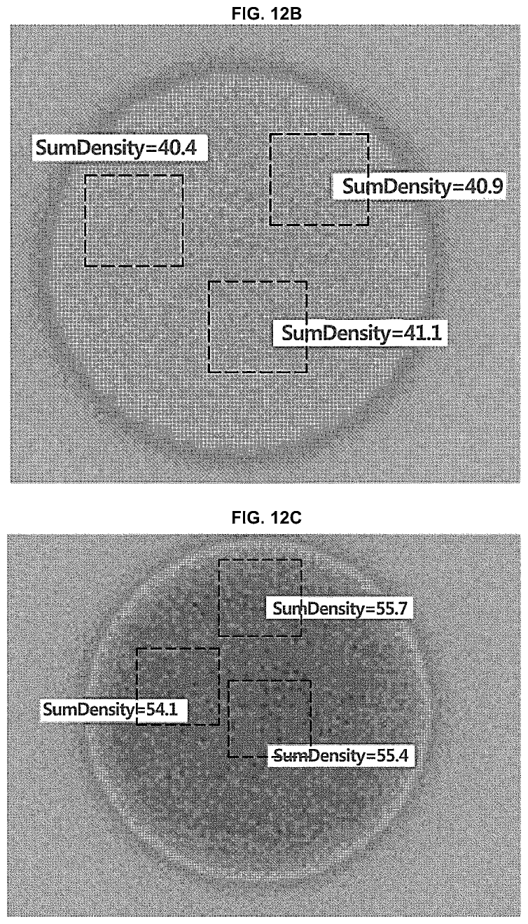

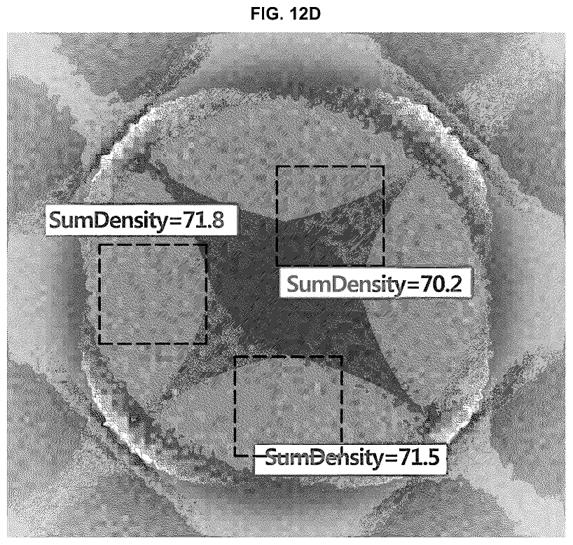

FIGS. 12A to 12D show the beam uniformity measurement results with the changes in power level inputted to the blue light irradiation device 100 according to an embodiment of the present disclosure.

Referring to FIGS. 12A to 12D, when the power level is 10%, 30%, 60%, 80%, the luminance at a plurality of areas of the blue light beam irradiated by the blue light irradiation device 100 may be measured.

In this experimental example, the luminance of three parts of the beam is measured at each power level. As shown in FIGS. 12A and 12B, even when the power level changes, the blue light beam irradiated by the blue light irradiation device 100 of the present disclosure shows a luminance difference of about 1-3%, and thus it can be seen that light uniformity of the beam is good.

Additionally, as the power level increases, the light intensity increases and the luminance also increases, and accordingly the light amount increases, and this can be seen numerically and through a change in chroma of blue light (as the power level increases, it becomes darker).

[Detailed Description of Main Elements]

| 10: Light source | 20: Control unit |
|---|---|
| R1, R2, R3, R4, R5, R6, R7, R8: Test area | |
| S: Skin area | |
| 100: Blue light irradiation device | 110: Light source unit |
| 111, 401: Coupler | 113: Female connector |
| 120: Cable | 130: Light irradiation unit |
| 131: Opening | 140: Fixation unit |
| 150: Support unit | 210: Control unit |
| 220: Cover | 230: Light source array |
| 231: Light source | 233: Cooling fan |
| 403: Male connector | 501: Holder |

1.-12. (canceled)

13. A blue light irradiation device for irradiating a plurality of blue light beams, comprising:
a plurality of light sources to emit blue light and a plurality of glass fibers each connected to each light source;
a light irradiation unit connected to each of the plurality of glass fibers, and including a plurality of openings each for irradiating a concentric blue light beam; and
a control unit to control power supplied to the plurality of light sources,
wherein each of the plurality of light sources include a plurality of light emitting diodes (LEDs).

14. The blue light irradiation device according to claim 13, wherein the control unit controls a light amount of each of the plurality of blue light beams by adjusting either power supplied to each of the plurality of light sources or irradiation time, or both.

15. The blue light irradiation device according to claim 13, wherein each of the plurality of light sources is configured to irradiate light with peak wavelength of 456 nm and full width at half maximum (FWHM) of 21 nm.

16. The blue light irradiation device according to claim 13, wherein the plurality of blue light beams irradiated through each of the plurality of openings is simultaneously irradiated with each separate light intensity, irradiation time and irradiation type.

17. The blue light irradiation device according to claim 16, wherein the irradiation type is a pulse beam or continuous beam type.

18. The blue light irradiation device according to claim 13, wherein the light irradiation unit includes six openings each for irradiating the blue light beam, and each of the plurality of light sources includes four mono-wavelength LEDs.

19. The blue light irradiation device according to claim 13, further comprising:
a heat sink and a cooling fan disposed around the plurality of light sources.

20. The blue light irradiation device according to claim 13, wherein each of the plurality of blue light beams irradiated through each of the plurality of openings is such that blue light emitted from the plurality of LEDs is irradiated in a form of a concentric beam having a luminance difference of a reference value or less through each of the plurality of glass fibers.
